(19) United States
(12) Patent Application Publication
(10) Pub. No.: US 2018/0071466 A1
(43) Pub. Date: Mar. 15, 2018

(54) WRIST BAND STRUCTURES FOR CONVENIENT ACCESS TO VITAL MEDICAMENTS

(71) Applicants: GWENDOLYN WHITE, ALTADENA, CA (US); JOHN KENNEDY, ALTADENA, CA (US); WES KENNEDY, LOS ANGELES, CA (US)

(72) Inventors: GWENDOLYN WHITE, ALTADENA, CA (US); JOHN KENNEDY, ALTADENA, CA (US); WES KENNEDY, LOS ANGELES, CA (US)

(21) Appl. No.: 15/261,240

(22) Filed: Sep. 9, 2016

(51) Int. Cl. A61M 15/00; A61M 15/08; A44C 5/00

(52) U.S. Cl. CPC A61M 15/0025; A44C 5/003; A61M 15/08; A61M 15/009

(57) ABSTRACT

Improved wrist band structures are provided for holding a fluid such as a vital medication within easy reach of the user. In one aspect, the unique flexible U-shaped elastic container may be used as an inhaler for relief of conditions such as asthma, allergies or the like. The flexible open-loop container may include an integral mouthpiece with frusto-conical nipple member for sipping the contained liquid from the container and for re-seal. In this form the mouthpiece is disposed on the curvature wall of the container at a location to permit easy access to the mouth of the wearer. The material for fabricating the unique bladder member of the wrist band may be selected from non-toxic water proof polymer materials, having sufficient elongation at break characteristics so that the inventive wrist band container may be easily slipped on the wrist, or over the hand, of the user but when once positioned cannot be easily displaced except by purposeful exertion. The bladder may also comprise an internal deformable support to assist shape retention of the wristband. The present invention thus provides a convenient, quick fluid dispensing device close at hand for a wide variety of users including the elderly and infirmed.

### BACKGROUND OF THE INVENTION

The present invention relates to a unique wristlet container for fluids and gels and more particularly, to an improved single use disposable container preferably fabricated from sterile elastomeric water proof material for holding medicinal and other liquids within easy reach and use by the wearer.

Those skilled in the art are generally familiar with a host of wrist band structures designed for a variety of purposes.

U.S. Pat. No. 2,127,926 discloses a wristlet device to be worn for instance, during certain games such as tennis to prevent the flow of perspiration from the arm into the palm of the hand of the wearer. In one embodiment, the wristlet shown consists of a relatively wide and thick band of flexible porous sponge rubber. As stated, the sponge rubber is capable of readily absorbing liquids and is a material from which liquids may be expelled by compression of the same. Although recognizing the virtues of convenient accessibility of a wearer's wrist for the disclosed device, the invention of Mason is designed to absorb liquids rather than to dispense liquids through the use of a elastic beverage container.

Kennedy, in U.S. Pat. No. 2,604,629, discloses a wrist band accessory for gloves. A stated object of the Kennedy invention is to provide an ornamental, elastic, fabric wrist band of decorative construction which may be worn in association with gloves to enhance the appearance of the wearer.

Cabernoch in U.S. Pat. No. 4,640,425 discloses a one-piece nursing container with means for storing nipple fabricated from a single web of flexible material. As stated, the container maintains the liquid and an attached nipple or other access assembly is provided in a sterile condition until use. Cabernoch recognizes the benefits of utilizing a pre-filled, pre-sterilized disposable container for routine feedings in hospitals and other institutional environments. For instance, the use of such containers is stated to avoid the time and effort necessary for bottle preparation, clean-up, washing and sterilization. The nursing containers disclosed in the Cabernoch patent, however, must be separately carried by the user which would generally interfere with various activities and are cumbersome to use.

Seward, in U.S. Pat. No. 4,818,544 discloses a system for obtaining beverages from sealed sachets containing a product containing a beverage when mixed with water for example, ground coffee or tea. In one embodied form, the Seward containers comprise an improved beverage sachets wherein the lands generally provide a more consistent and better opening of a sachet when used.

Other known wrist apparel inventions are disclosed in U.S. Pat. No. 4,067,063, inventor Donald Ettinger; U.S. Pat. No. 1,534,208, inventor Delsworth E. Gibson; U.S. Pat. No. 5,305,470, inventor William D. McKay; U.S. Pat. No. 5,119,513, inventor William D. McKay; U.S. Pat. No. 4,517,685, inventor Gary M. Lesley; and U.S. Pat. No. 4,462,116, inventor Salvatore J. Sanzome and Donald Krost.

Other improved packages for beverages and the like are disclosed in U.S. Pat. No. 5,203,459, inventor Leslie Wade; U.S. Pat. No. 4,803,205, inventor John Hammond and Leonard Reed; U.S. Pat. No. 4,765,514, inventor Albert I. Berglund.

In U.S. Pat. No. 5,653,732 to Sheehy, there is disclosed a nipple element for drinking contained liquids of a “natural form” design. The nipple has an annular rim, a lower segment, an intermediate segment, an upper segment and a tip. The annular rim is used as a securing structure and is adjacent to, and integrally formed with, the lower segment having a large curved outer surface. The lower segment is adjacent to, and integrally formed with, the intermediate segment that has a smaller curved outer surface and is smaller than the lower segment. The intermediate segment is adjacent to, and integrally formed with, the upper segment that has a smaller curved outer surface than the intermediate segment. The upper segment is adjacent to, and integrally formed with, the tip. However, the disclosed nipple suffers from the drawback of having three segments or areas that do not simulate the shape and function of a woman's breast.

Accordingly, those skilled in the art have recognized a significant need for more convenient container for fluid or gel medicaments such as angina or asthma medicines which will provide the user with a simple effective device for ingesting or inhaling the medication in a convenient manner close at hand. The present invention fulfills these needs.

### SUMMARY OF THE INVENTION

Several objectives and advantages of the invention are attained by the various embodiments and related aspects of the invention summarized below.

Improved wrist band structures are provided for holding a fluid such as vital medication within easy reach of the user. In one embodied form the inventive wristband may be fabricated as a flexible U-shaped elastic container that may be used for ingesting or inhaling medications for relief of asthma, allergies or the like. In a preferred embodiment, the unique wristband comprises an integral mouthpiece such as a re-sealable straw or frusto-conical nipple member for sipping the contained liquid from the wristband. In this embodied form the mouthpiece is disposed on the upper outer wall portion of the container facing the user to permit easy access to the wearer's mouth. The medical grade elastic material for fabricating the unique wrist band may be selected from water proof materials, having sufficient elongation at break characteristics so that the inventive wrist band may be easily slipped on or over the wrist, but when once positioned cannot be easily displaced except by purposeful exertion. The interior of the wristband bladder may further comprise an absorbent sponge material or contain a gel ringlet to assist shape retention of the bladder on the wearer's wrist. The present invention thus provides a convenient, quick fluid, gel or vapor dispensing device close at hand for a wide variety of users including the elderly and infirmed.

In a presently preferred embodiment, the invention provides an elastic single use wrist band fluid container that can be conveniently worn by the user. The container may take the form of a horseshoe shaped flexible bladder that comprises two arcuate sections or wings to conform to the user's wrist. The bladder member may be single or double walled fabricated from a suitable polymer material of sufficient elongation at break to engage, and to remain in position on at least a portion of the wearer's wrist. A mouthpiece element is disposed on an upper portion of the wristband wall facing the wearer to permit the user to conveniently ingest, or inhale, at least a portion of the fluid contents from the container. In a preferred implementation, the mouthpiece closure is a nipple or straw operatively connected to the bladder member enabled to be moved between (i) a closed position in which the contents of the container is secure and (ii) an open position in which access and egress to the contained liquid, or vapor, is provided. In another implementation, the closure may include a re-sealable strap, or tape.

In another related embodiment, the invention provides an inter-changeable mouthpiece or inhaler for fluid or mist delivery system such as a straw or conduit that extends from the container. The conduit may be pop-up or extended by the user in a conventional manner for instance known with juice packs. In another implementation, the bladder comprises a distended nipple closure that may be inserted into the user's nostril for inhalation of the contained medicament. In either form, the closure may include a re-sealable adhesive foil tape, or a removable screw cap for convenient opening and re-sealing of the bladder member.

Preferably, the mouthpiece will be fabricated as an integral nipple element simulating the shape, surface geometry and function of a woman's breast is utilized. The nipple comprises a stem and a base. The base has an areola region and a bulbous region. The areola region is positioned between the stem and the bulbous region, and can simulate the areola of a woman's breast. The bulbous region can simulate the region of a woman's breast surrounding the areola. The areola region has a texture or surface geometry that is different from the texture or surface geometry of the stem or bulbous region.

In yet another embodiment, the bladder member may comprise a reinforcing plastic or flexible metal spine with memory shape characteristics. The spine is designed and constructed to assist the secure placement and positioning of the bladder member on the wrist of the user. The mouthpiece portion can be any of a number of different varieties, sizes and shapes.

Accordingly, those skilled in the art have recognized a significant need for more convenient fluid containers which will provide the user with a simple effective device for providing a wide variety of potable liquids in a convenient manner. The present invention fulfills these needs.

### BRIEF DESCRIPTION OF THE DRAWING

FIG. 1 is a front perspective view of the unique improved wrist band for medicaments in accordance with one embodied form of the present invention being worn by a user;

FIG. 2 is a front perspective view of a second embodied wrist band for medicaments in accordance with the present invention;

FIG. 3 is a cross sectional view of third embodied wrist band for medicaments in accordance with the present invention depicting a mouthpiece portion without closure element;

FIG. 4 is a cross-sectional view of yet another embodied form of the wrist band for portable liquids in accordance with the present invention depicting a mouthpiece portion having a distended nipple closure element;

FIG. 5 is a side view of an embodied pop-up nipple closure for the improved wrist band for potable liquids in accordance with the present invention;

FIG. 6 is a side view of yet another embodied U-shaped bladder member of the inventive wrist band with reinforced distal ends and without closure element;

FIG. 7 is a side view of an embodied wrist band with a generally circular bladder member having a closure element with integral conduit mouthpiece;

FIG. 8 is a perspective view of another embodied inventive wristband having a mouthpiece with removable cap closure element; and

FIG. 9 is a perspective view of an embodied wristband having a mouthpiece with removable cap exposing a distended nipple element for access to the contained medicament.

### DETAILED DESCRIPTION OF THE PREFERRED EMBODIMENTS

Improved wrist band structures are provided for holding a fluid, gel or vapors such as vital medication within easy reach of the user. In one embodied for the unique flexible U-shaped elastic container may be used as an inhaler for relief of conditions such as angina, asthma, allergies or the like. In a preferred embodiment, the unique flexible open-loop container may comprise an integral mouthpiece such as a frusto-conical nipple, or pop-up straw for sipping the contained liquid from the container.

In this embodied form the mouthpiece is preferably disposed on the upper curvature wall facing the user, to permit easy access to the mouth of the wearer. The material for fabricating the unique wrist band may be selected from medical grade elastic and water proof materials, having sufficient elongation at break characteristics so that the inventive wrist band container may be easily slipped on the wrist over the hand of the user but when once positioned cannot be easily displaced except by purposeful exertion.

In this respect, the elongation at break, also known as fracture strain, is the ratio between changed length and initial length after breakage of the test specimen. It expresses the capability of the polymer to resist changes of shape without crack formation. Suitable polymers will have an elongation at break from about 10 percent to about 75 percent, and preferable from 30 to 60 percent. The elongation at break is determined by tensile testing in accordance with EN ISO 527. The present invention thus provides a convenient, quick fluid dispensing device close at hand for a wide variety of users including the elderly and infirmed.

Preferably when used for inhaling vaporized medicines such as nitroglycerine for relaxing or widening of the blood vessels for prevention or relief of chest pain, the bladder comprises an deformable support element such as internal ringlets or interior medical grade foam to minimize the potential for collapse of the bladder walls. A foam or sponge element may include one or more semi-permeable membranes to control the flow of mist from the bladder. Other known vaporizing medicines include Camphor such as used in Vick's Vaporub™, Benzrdrex™ propylhexedrine, and vaporized botanicals suitable for dispensing, for instance, by squeezing, a portion of the wristband.

The unique wrist band may also be used as a liquid or mist dispenser in the treatment of asthma or allergies. Many allergies require quick relief that may be administered by nasal inhaler. In this respect, the mouthpiece member disposed on the bladder may be replaced by a nasal inhaler conduit or used with a portable nebulizer. A nebulizer changes medication from a liquid to a mist so that it Can be more easily inhaled into the lungs. Portable nebulizers may run on batteries, and carried in a purse or pocket to be used whenever or wherever the need arises.

The deformable support for the inside of the bladder can be made from any suitable medical grade substance such as foam, sponge, gauze, non-woven sponge, cellulose filled sponges and the like.

Additionally, the bladder may include internal, external, or embedded reinforcing ribs and/or reinforcing spines to limit elongation of the polymer and/or provide support for the bladder at any desired area.

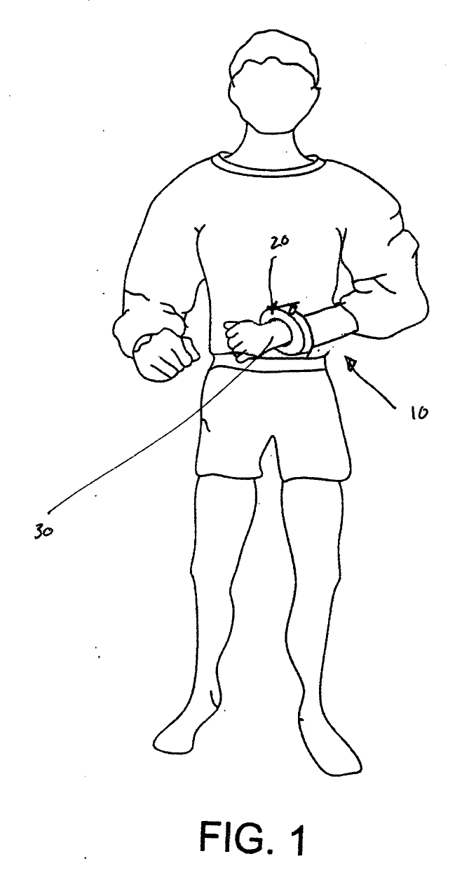

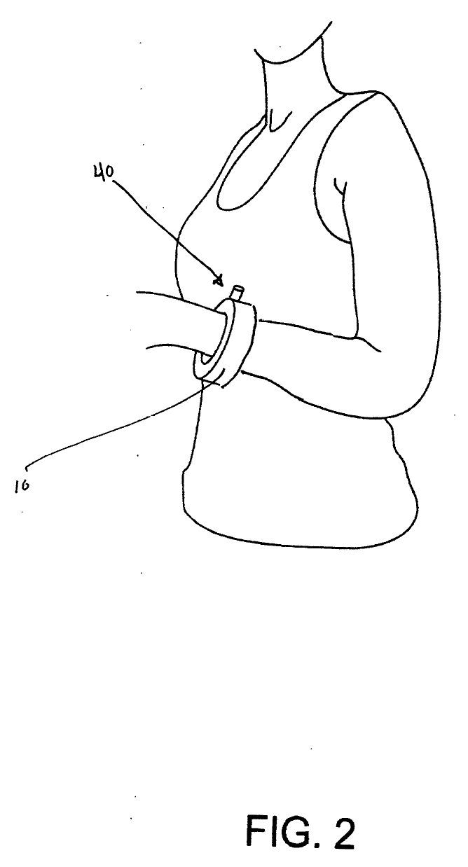

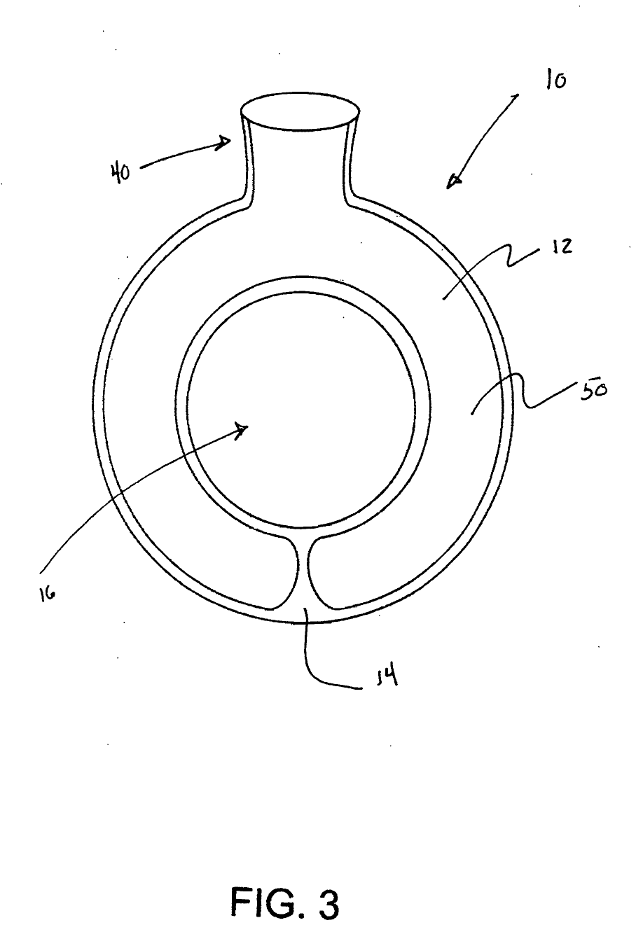

Referring now to the drawings, FIG. 1 illustrates an improved wrist band 10 for vital medicaments in accordance with one embodiment of the present invention. The wrist band 10 comprising an annular elastic container 20 for maintaining an ingestible medicine or the like to be worn on the wrist 30, of a user. In a second embodiment, shown most clearly in FIGS. 2, 4, and 9, the unique flexible annular container may further comprise a distended nipple element 40 for inhalation of the medicament to be dispensed from the container. Accordingly, in this embodied form the nipple closure 40 is disposed at a top portion of the outer wall facing the user to permit easy access to the nasal area of the wearer. Any conventional nasal inhaler devices such as those disclosed in U.S. Pat. No. 3,939,751 issued to Birch et al and U.S. Pat. No. 4,013,075 issued to Cocozza may be accordingly modified to be used with the disclosed wrist band structures.

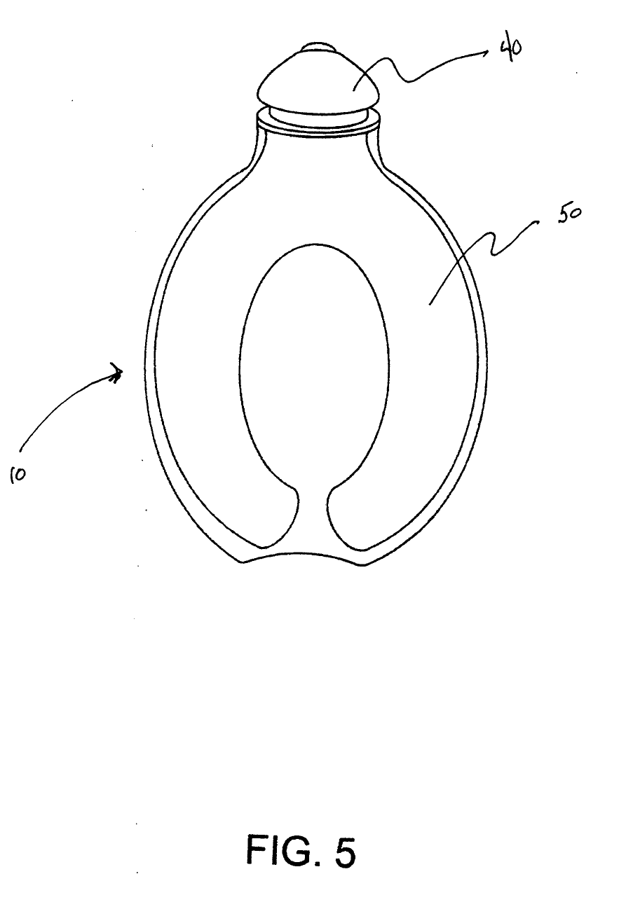

The elastomeric material for fabricating the bladder of the unique wrist band may be selected from non-toxic water proof materials, having sufficient elongation at break characteristics so that the inventive container may be easily slipped on the wrist over the hand of the user but when once positioned cannot be easily displaced except by purposeful exertion. To maintain the desired shape of the bladder 50, an internal, external, or molded deformable support features may be utilized. As shown in FIG. 6, the distal ends 60a and 60b of the arcuate shaped bladder 50, may be reinforced by molded ridges 70 disposed on the portions of the bladder 50. Of course, such reinforcement may be disposed on any selected area of the bladder 50.

The present invention thus provides a convenient, quick fluid and mist dispensing device for a wide variety of users including those suffering from treatable ailments as well as for the elderly and infirmed.

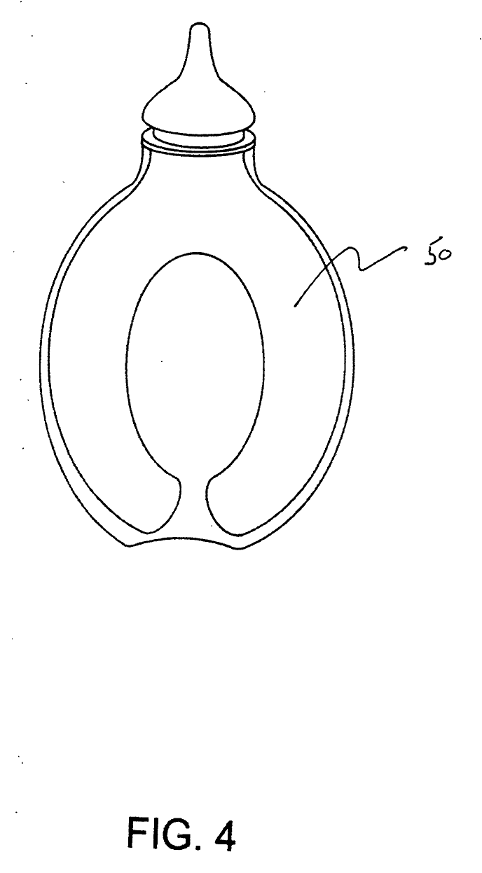

In more detail, and as shown in FIG. 1, the improved wrist band 10 for liquid medications may be include an integral or removable mouthpiece 80 for sipping or ingesting the contained fluid. FIG. 5 depicts a preferred nipple closure 90 having one or more small slits disposed at the top 92 providing access and egress to the contained medication. As further shown in this sectional view the bladder 50 comprises a curvilinear deformable support 94.

Typically, the wrist band 10 will be fabricated from stretchable inert plastic materials such as rubber, polymers and copolymers, having sufficient elongation to break characteristics and liquid impermeability to permit the wrist band to be stretched over the hand, but once in position, to maintain a snug placement on the wrist of the wearer. The materials for fabricating the inventive wrist band must also be non-toxic and is preferably bio-degradable.

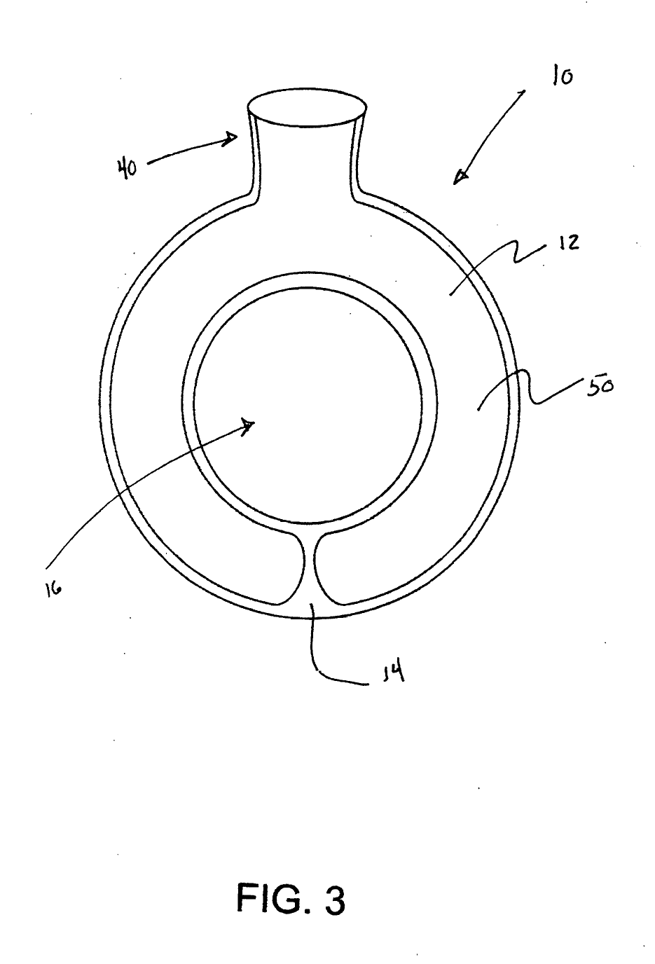

As shown in FIG. 3, the inventive wrist band 10 may be doubled walled with an open loop internal bladder 12 and a generally concentric outer wall 14. Preferably the annulus diameter 16 will provide from one to two inches of clearance when positioned on the wrist of a wearer.

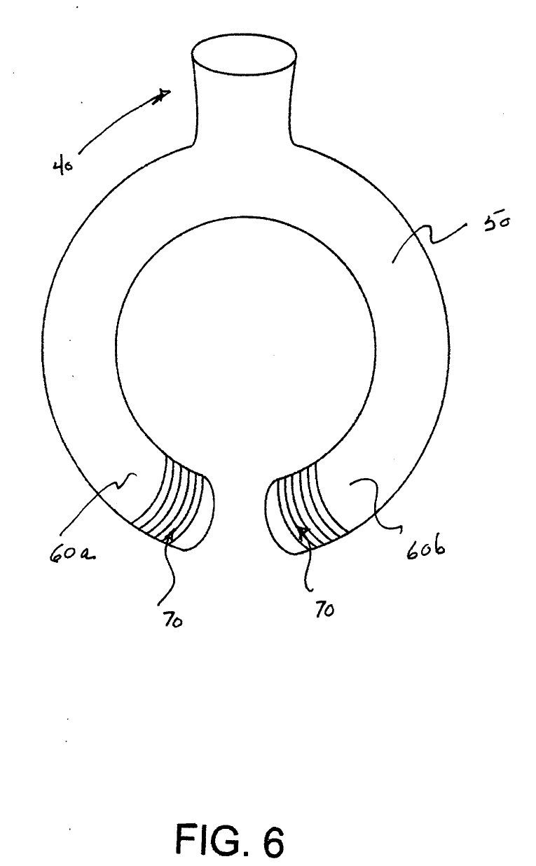

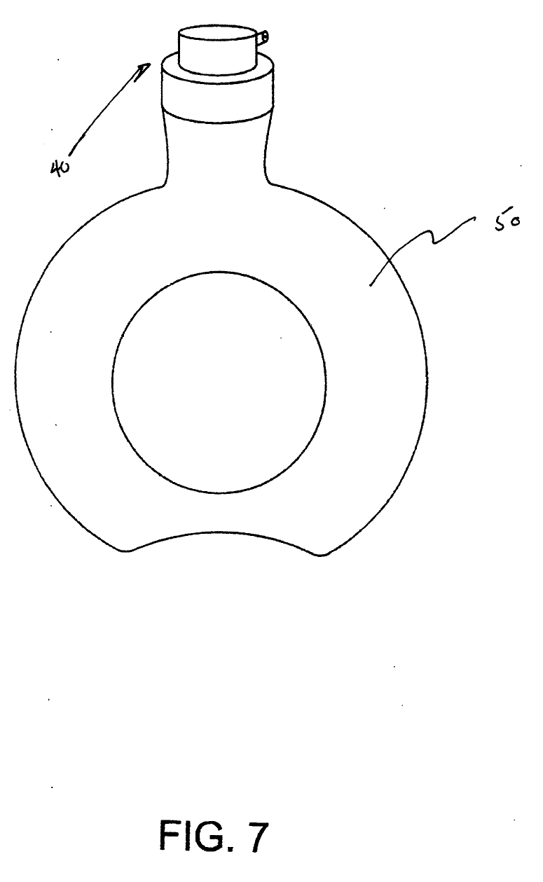

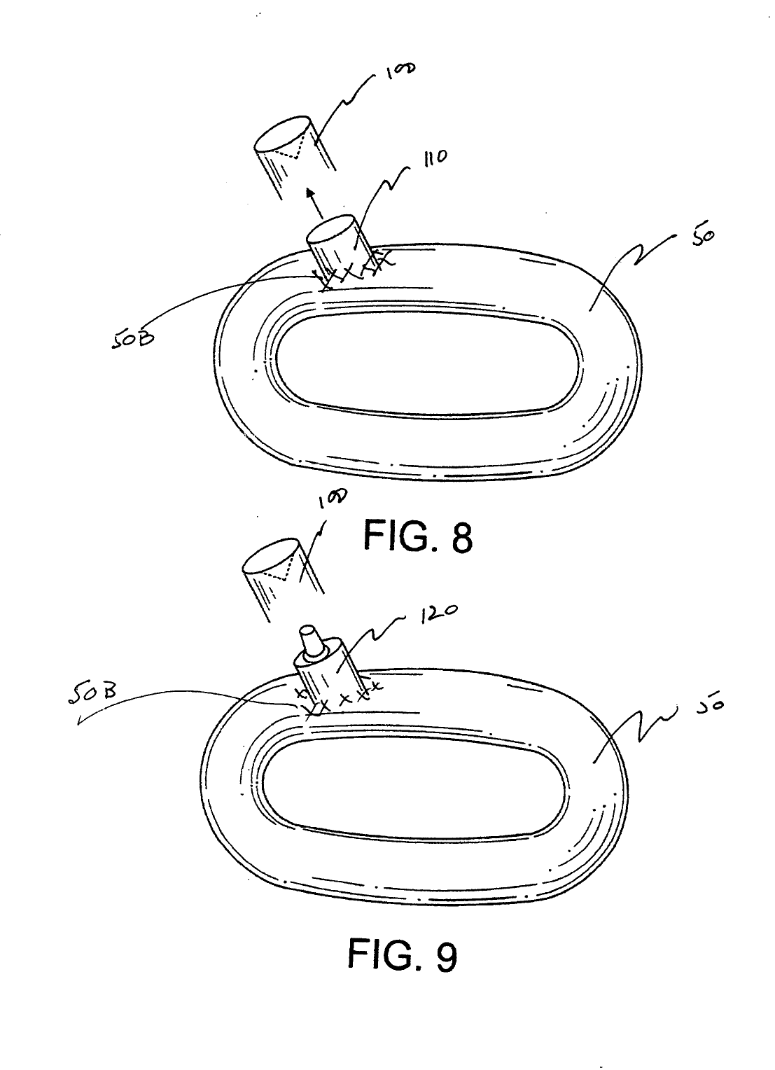

The preferred closure member for liquid medications a nipple, such as disclosed in U.S. Pat. Nos. 4,818,544 and 4,830,205 dispensing of the fluid from the bladder 50 such as shown in FIGS. 7 and 8. The closure may be accomplished by other means, such as by re-sealable foil tab or with a straw. As shown in FIGS. 8 and 9 the mouthpiece or nasal inhaler closure may be a removable or screw cap 100 to re-seal the fluid conduit 110 or 120 after use. Each fluid conduit will preferably be disposed on the outer wall 50 of the bladder on the upper portion in a position that permits easy access to the mouth of the wearer. The bladder 50 may also be provided with reinforced areas 50B such as stiffening ribs to ensure a liquid tight seal and proper orientation of the fluid conduit 110 or 120.

The unique wristbands of the present invention can be manufactured by any conventional technique to produce:
Those with double walled bladders;
Those with single walled bladders; and
Those with mouthpiece members or nasal inhalers.

Suitable materials for fabricating the bladder member are preferably medical grade polymers in different grades, thicknesses, textures, and colors. The closure elements may comprise pop off-pull off-peel off caps; harder straws, and different types of nipples with new shapes and textures. The following information is provided to assist the skilled artisans to under the invention disclosed and the environment in which it will typically be used.

Countless medical procedures are conducted throughout the world on a daily basis. A myriad of different fluids maybe administered to a patient to serve a variety of different purposes: feeding fluids to nourish parenterally, saline to hydrate, analgesics to suppress pain, therapeutic drugs to treat disease, contrast media to enhance images of organs and other internal bodily structures, etc.

The unique wrist band of the present invention may be a water winged type of inner tube which would spread apart at the bottom when in use. This would give a less bulky feeling at the wrist area, the outer band, covering the same area at the wrist, could then be redesigned to compliment the inner tube, for a more comfortable feel.

The wristband with a nipple, containing both an inner tube which implodes in a similar way to an inner liner in a baby's bottle, will be helpful and more comfortable for babies, young children, the elderly and infirmed. The original idea of course, was to keep babies from swallowing air.

For generations baby bottles worked without inner lines a mother would simply tap the nipple to be sure it was completely filled with milk, and then hold the bottle at an angle with hopes that air would not get into the baby's stomach.

Those skilled in the art, will appreciate that ‘a single or multiple piece mini inner tube is provided, having an integral suction device (nipple) etc., etc’, a water winged type of inner tube could be professionally designed to include the nipple, and the outer tube would be a medical cover designed to be somewhat flatter at the wrist area in addition to being protection for the inner tube, the outer tube having a designed extension which, when wrapped around the bottom of the nipple, resembles a soft lip shaped base. The size of both the nipple and lip shaped base depend upon the users age. Both adult and children's nipples might be covered by using a pull off “shaped seal” which will be manufactured to fit and be used several times.

Medical wristbands with our unique closure elements will be of great help to people all over the world who suffer with asthma and/or allergy related problems. This having been said, some patients needing inhaled types of medications will need a slightly pressurized type of wristband with a different delivery system, such as aerosol.

The ‘pressure inhaler tube’ is a single tube similar to the embodiments shown in FIGS. 8 and 9. The basic idea behind the inhaler tube is to use a medical wristband in a similar way to over the counter plastic inhaler bottles, or the like, found in most drug stores. The buyer buys a plastic bottle with a nose straw attached, or a larger capped mouth piece-takes it home-unscrews the cap and uses hand pressure to squeeze a spray mist, or the like, into the affected area, wherever it may be. Therefore; the pressure inhaler band will be made of stronger transfusion bag material than bands made with nipples.

The straw may have an oval shaped mouth piece or a reed like shape, similar to a flexible cane or a metal piece which is attached to the mouth of a wind instrument, or it might have a similar shape to the mouth piece of a whistle. Whatever shape the straw may take, is see a lip shaped mouth rest circling it's' bottom.

The straw like mouth piece will not be placed at the center of the wrist, but near the edge of the wrist, directly below the thumb. A right handed person wearing the inhaler tube on the right hand, would use his/her left hand to reach under and around the tube to pressure squeeze the medical mist or liquid out into his/her mouth.

The wrist band will preferably contain two to six fluid ounces of the desired medicament. Accordingly, an inventive single or multiple piece “mini-inner tube” is provided having an integral suction device (nipple) providing means for dispensing the fluid contained within the wrist band. The band may either be reusable or disposable and can also provide a suitable substrate for indicia, such as advertising and/or directions for use.

We claim:

1. An improved wrist band structure containing a vital liquid medicament within easy reach of a user, said wrist band comprising an elastic bladder having an upper portion, a lower portion, and intermediate curvilinear sidewalls; said bladder being water-proof and composed of stretchable resilient, polymeric material having elongation at break within a range of 10 to 75 percent to permit said elastic container to be easily slipped on the wrist, over the hand of a wearer, but when once positioned cannot easily be displaced except by purposeful exertion; a dispensing member disposed on the upper portion of the bladder container facing the user, for access and egress of the liquid medicament from the container; and a support element for minimizing the potential for collapse of said bladder walls.

2. The improved wrist band as defined in claim 1, wherein the bladder has a substantially annular shape.

3. The improved wrist band as defined in claim 1, wherein the bladder has an open-loop shape.

4. The improved wrist band as defined in claim 1, wherein the dispensing member comprises a nipple.

5. The improved wrist band as defined in claim 1, wherein the dispensing member comprises a fluid conduit.

6. The improved wrist band as defined in claim 1, wherein the dispensing member is integral with said bladder member.

7. The improved wrist band as defined in claim 1, wherein said bladder member comprises reinforcing ribs adjacent said dispensing member.

8. The improved wrist band as defined in claim 1, wherein said support element is deformable.

9. The improved wrist band as defined in claim 1, wherein said support element is internally mounted in said bladder member.

10. The improved wrist band as defined in claim 1, wherein said support member is integral with said bladder member.

11. The improved wrist band as defined in claim 1 wherein said support member comprises a reinforcing spine integral with said bladder member.

12. The improved wrist band as defined in claim 1 wherein said dispensing member comprises a nasal inhaler.

13. The improved wrist band device as defined in claim 1 wherein said medicament is selected from the group consisting of: asthma medicaments, allergy medicaments, and angina medicaments.

14. The improved wristband as defined in claim 1 wherein the contained medicament is a gel.

15. The improved wrist band as defined in claim 1, wherein the contained medicament can be dispensed as a mist.
